(19) United States
(12) Patent Application Publication
(10) Pub. No.: US 2022/0355071 A1
(43) Pub. Date: Nov. 10, 2022

(54) CATHETER AND METHOD FOR PRODUCING THE CATHETER

(71) Applicant: KANEKA CORPORATION, Osaka-shi, Osaka (JP)

(72) Inventors: Masaki TAKEMURA, Okaya-shi (JP); Toshiya KISA, Okaya-shi (JP)

(73) Assignee: KANEKA CORPORATION, Osaka-shi, Osaka (JP)

(21) Appl. No.: 17/621,309

(22) PCT Filed: Apr. 13, 2020
(86) PCT No.: PCT/JP2020/016267

§ 371 Date: Dec. 21, 2021

(30) Foreign Application Priority Data
Jun. 25, 2019 (JP) 2019-117003

(51) Int. Cl. A61M 25/00; A61B 5/00; A61B 18/14; A61B 5/287; B29C 65/48; B29C 65/00

(52) U.S. Cl. CPC A61M 25/0052; A61B 5/6852; A61B 18/1492; A61M 25/0009; A61B 5/287; A61M 25/0015; B29C 65/48; B29C 66/53; B29C 66/03; A61B 2018/00595

(57) ABSTRACT

This catheter has: a shaft (2) that has a first end and a second end in the longitudinal direction thereof, has a lumen (3) extending in the longitudinal direction, and has formed therein a side hole (4) being in communication with the lumen (3); a protective tube (10) that is inserted in the side hole (4); a first electrode (20) that is disposed outside the shaft (2); and a wire (30) that is electrically connected to the first electrode (20), passes through the inside of the protective tube (10), and extends into the lumen (3) of the shaft (2).

### TECHNICAL FIELD

The present invention relates to an electrode catheter used for measuring a potential of an organ in the body, mainly a cardiac potential, or cauterizing a tissue in the body, and a method for producing the electrode catheter.

### BACKGROUND ART

An electrode catheter is a medical instrument mainly used to diagnose arrhythmia by measuring the cardiac potential or to cauterize a body tissue by passing a high-frequency current to treat the arrhythmia. The electrode catheter has a plurality of electrodes outside a shaft having a lumen. A wire electrically connected to the inner surfaces of the electrodes extends through the lumen of the shaft to an electrocardiograph. The wire is connected to the electrocardiograph using a connector. With this configuration, it is possible to accurately confirm the state of the myocardium that causes the arrhythmia by, for example, inserting the electrode catheter into the heart of a patient, connecting the connector to the electrocardiograph, and measuring the electrocardiogram in the vicinity of the electrode portion. For example, Patent Document 1 discloses a catheter having an electrode for direct current tissue therapies.

### RELATED ART DOCUMENTS

### Patent Documents

Patent Document 1: JP-A-2014-502195

### SUMMARY OF THE INVENTION

### Technical Problem

A method for producing the catheter having an electrode as described above commonly includes preparing a shaft having a side hole and a wire to which the electrode is connected, and inserting the wire into the side hole of the shaft. In this method, the inner wall forming the side hole of the shaft and the wire come into contact with each other during insertion of the wire, which may not allow smooth insertion of the wire into the lumen of the shaft. Thus, there is room for improvement. In view of this, an object of the present invention is to provide a catheter that facilitates insertion of a wire into a shaft, and a method for producing the catheter.

### Solutions to the Problems

The gist of one embodiment of a catheter of the present invention is according to the present invention that can overcome the above problems is as follows. The catheter includes: a shaft having a first end and a second end in a longitudinal direction and a lumen extending in the longitudinal direction, the shaft having a side hole that communicates with the lumen; a protective tube inserted in the side hole; a first electrode disposed outside of the shaft; and a wire electrically connected to the first electrode and extending in the lumen of the shaft through the protective tube. According to the catheter described above, the protective tube is inserted into the side hole of the shaft, so that the shaft and the wire are less likely to come into contact with each other during insertion of the wire, whereby the wire is easily inserted into the shaft. In addition, since the wire can be smoothly inserted into the shaft in this manner, kink of the wire and damage to the wire can also be prevented.

Preferably, the protective tube has a first end and a second end in a longitudinal direction of the protective tube, the protective tube has a first opening at the first end of the protective tube and a second opening at the second end of the protective tube, the first opening is located radially outward of the shaft than the second opening, and a proximal end of the second opening is located proximal to a proximal end of the first opening.

Preferably, the protective tube extends along a radial direction of the shaft.

Preferably, the protective tube has a first end a second end in a longitudinal direction of the protective tube, the protective tube has a first opening at the first end of the protective tube and a second opening at the second end of the protective tube, the first opening is located radially outward of the shaft than the second opening, and at least a part of the second opening is located radially inward of the shaft than an inner surface of the shaft.

Preferably, the protective tube has a first end and a second end in a longitudinal direction of the protective tube, the protective tube has a first opening at the first end of the protective tube and a second opening at the second end of the protective tube, the first opening is located radially outward of the shaft than the second opening, and an inner adhesive is disposed in the protective tube on the first opening side.

Preferably, the inner adhesive is disposed between an inner surface of the first electrode and an end surface of the first end of the protective tube.

Preferably, further comprising an outer adhesive disposed between an inner wall forming the side hole of the shaft and an outer surface of the protective tube, the outer adhesive being disposed on at least a part of a circumferential direction of the protective tube.

Preferably, the outer adhesive is disposed around an entire circumference of the protective tube.

Preferably, the outer adhesive is disposed on the outer surface of the protective tube and located radially inward of the shaft than the inner surface of the shaft.

Preferably, the protective tube has a first end and a second end in a longitudinal direction of the protective tube, the protective tube has a first opening at the first end of the protective tube and a second opening at the second end of the protective tube, the first opening is located radially outward of the shaft than the second opening, an inner adhesive is disposed in the protective tube on the first opening side, and the inner adhesive and the outer adhesive are made of a same material.

Preferably, the outer adhesive has a first outer adhesive and a second outer adhesive made of different materials, the first outer adhesive is disposed on a first section in the circumferential direction of the protective tube, and the second outer adhesive is disposed on a second section other than the first section in the circumferential direction of the protective tube.

Preferably, the protective tube has a first end and a second end in a longitudinal direction of the protective tube, the protective tube has a first opening at the first end of the protective tube and a second opening at the second end of the protective tube, the first opening is located radially outward of the shaft than the second opening, an inner adhesive is disposed in the protective tube on the first opening side, and the inner adhesive and one of the first outer adhesive and the second adhesive are made of a same material.

Preferably, an outer surface of the first electrode is disposed on a same plane as an outer surface of the shaft or disposed inward of the outer surface of the shaft in the radial direction of the shaft.

Preferably, an angle between a central axis of the side hole and a longitudinal direction of the shaft is an acute angle.

Preferably, a length of a portion of the protective tube disposed radially inward of the shaft from the inner surface of the shaft is longer than an inner diameter of the shaft.

Preferably, the shaft has a reinforced section and a non-reinforced section in the longitudinal direction of the shaft, a reinforcing material made of metal is disposed in the reinforced section, the non-reinforced section is located distal to the reinforced section, and the reinforcing material is not disposed in the non-reinforced section, the first electrode is disposed in the reinforced section, and the second electrode is disposed in the non-reinforced section and disposed more distally than the first electrode.

The gist of one embodiment of a method for producing the catheter according to the present invention that can overcome the above problems is as follows. The method for producing a catheter comprising: making a side hole communicating with a lumen in a shaft having a first end and a second end in a longitudinal direction, the lumen extending in the longitudinal direction; inserting a protective tube into the side hole; applying an outer adhesive to an outer surface of the protective tube; inserting a wire electrically connected to a first electrode into the protective tube; inserting an inner adhesive into the protective tube; and disposing the first electrode on the side hole. According to the method for producing the catheter described above, the protective tube is inserted into the side hole of the shaft, so that the shaft and the wire are less likely to come into contact with each other during insertion of the wire, whereby the wire is easily inserted into the shaft. In addition, since the wire can be smoothly inserted into the shaft in this manner, kink of wire and damage to the wire can also be prevented.

Preferably, further comprising adjusting a direction of a central axis of the side hole before inserting the protective tube into the side hole.

Preferably, further comprising removing at least a part of a portion of the protective tube protruding from the shaft before inserting the wire into the protective tube.

Preferably, when inserting the protective tube into the side hole, the protection tube is pulled back by a second predetermined length shorter than a first predetermined length after inserting the protective tube by the first predetermined length.

### Effects of the Invention

According to the catheter of the present invention and the method for producing the same, the shaft and the wire are less likely to come into contact with each other during insertion of the wire, whereby the wire is easily inserted into the shaft.

### BRIEF DESCRIPTION OF THE DRAWINGS

FIG. 1 is a side view of a catheter according to an embodiment of the present invention.

FIG. 2 is an enlarged side sectional view of a portion II of the catheter illustrated in FIG. 1.

FIG. 3 is a side sectional view illustrating modification of the catheter illustrated in FIG. 2.

FIG. 4 is a side sectional view illustrating another modification of the catheter illustrated in FIG. 2.

FIG. 5 is a side sectional view illustrating still another modification of the catheter illustrated in FIG. 2.

FIG. 6 is a side sectional view illustrating still another modification of the catheter illustrated in FIG. 2.

FIG. 7 is a sectional view of the catheter illustrated in FIG. 5 along a line VII-VII.

FIG. 8 is a sectional view illustrating modification of the catheter illustrated in FIG. 7.

FIG. 9 is a sectional view illustrating another modification of the catheter illustrated in FIG. 7.

### MODE FOR CARRYING OUT THE INVENTION

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

An embodiment of the catheter of the present invention is characterized in that the catheter includes: a shaft having a first end and a second end in a longitudinal direction and a lumen extending in the longitudinal direction, the shaft having a side hole that communicates with the lumen; a protective tube inserted in the side hole; a first electrode disposed outside of the shaft; and a wire electrically connected to the first electrode and extending in the lumen of the shaft through the protective tube. According to the catheter described above, the protective tube is inserted into the side hole of the shaft, so that the shaft and the wire are less likely to come into contact with each other during insertion of the wire, whereby the wire is easily inserted into the shaft. In addition, since the wire can be smoothly inserted into the shaft in this manner, kink of the wire and damage to the wire can also be prevented.

A configuration example of the catheter will be described with reference to FIGS. 1 and 2. FIG. 1 is a side view of the catheter according to an embodiment of the present invention, and FIG. 2 is an enlarged side sectional view of a portion II of the catheter illustrated in FIG. 1. The catheter 1 includes a shaft 2, a protective tube 10, a first electrode 20, and a wire 30. The shaft 2 has a first end and a second end defining a longitudinal direction. The distal side of the catheter 1 and the shaft 2 refers to the first end side in the longitudinal direction (in other words, the direction of a longitudinal axis of the shaft) of the shaft 2, and refers to a treatment target side. The proximal side of the catheter 1 and the shaft 2 refers to the second end side in the longitudinal direction of the shaft 2 and the hand side of a user (operator). In FIG. 1, the left side represents the distal side, and the right side represents the proximal side. In the radial direction of the shaft 2, the inward direction refers to a direction toward the center of the longitudinal axis of the shaft 2, and the outward direction refers to a radial direction opposite to the inward direction.

The catheter 1 is used, for example, for diagnosis and treatment of arrhythmia. In the diagnosis of arrhythmia, the catheter 1 is inserted into the body of a patient so that the electrode is located near a tissue to be diagnosed of the heart, and the potential of the tissue is measured. Thus, an electrocardiogram can be obtained. In addition, in the treatment of arrhythmia, the body tissue can be cauterized by applying a high-frequency current to the electrode of the catheter 1, for example.

The shaft 2 is a member inserted into the body of the patient from the distal side. The shaft 2 has the first end and the second end in the longitudinal direction. The shaft has a lumen 3 extending in the longitudinal direction. The shaft 2 preferably has a tubular structure so that the wire 30 is placed in the lumen 3. In addition, the shaft 2 is inserted into the body, and thus, preferably has flexibility. Examples of the shaft 2 having a tubular structure includes: a hollow body constituted by arranging one or a plurality of wire rods in a predetermined pattern; a member obtained by coating at least one of an inner surface or an outer surface of the hollow body with a resin; a cylindrical resin tube; and a combination thereof, for example, a member obtained by connecting the above members in the longitudinal direction of the shaft 2. Examples of the hollow body constituted by arranging one or a plurality of wire rods in a predetermined pattern include a cylindrical body having a web structure in which the wire rods simply cross each other or the wire rods are interweaved, and a coil around which the wire rods are wound. As the wire rod, one or more single wires may be used, or one or more stranded wires may be used. The resin tube can be manufactured by, for example, extrusion molding. When the shaft 2 is a tubular resin tube, the shaft 2 can be composed of a single layer or a plurality of layers. The shaft 2 may be configured such that a part in the longitudinal direction or in the circumferential direction is composed of a single layer, and another part is composed of a plurality of layers. Although not illustrated, the shaft 2 may have a plurality of lumens 3. As illustrated in FIG. 1, a handle 40 gripped by the operator is preferably connected to the proximal side of the shaft 2.

The shaft 2 can be made of, for example, a synthetic resin such as a polyolefin resin (for example, polyethylene or polypropylene), a polyamide resin (for example, nylon), a polyester resin (for example, PET), an aromatic polyether ketone resin (for example, PEEK), a polyether polyamide resin, a polyurethane resin, a polyimide resin, or a fluororesin (for example, PTFE, PFA, ETFE), or metal such as stainless steel, carbon steel, or a nickel-titanium alloy. The above materials may be used singly, or two or more kinds of the materials may be used in combination.

Although not illustrated, a reinforcing material made of metal may be provided on the shaft 2. The reinforcing material may be in the form of laminate, or may be formed by arranging or braiding wire rods including a single wire or a stranded wire in a specific pattern. As a result, the strength and torque of the shaft 2 can be enhanced. The cross section of the wire rod may have, for example, a circular shape, an oval shape, a polygonal shape, or a shape obtained by combining these shapes. Note that the description of the metal constituting the shaft 2 can be referred to for a material constituting the reinforcing material. The reinforcing material can be provided on an outer surface 7 or an inner surface 6 of the shaft 2, or on the wall of the shaft 2.

The reinforcing material is preferably provided on at least a part of the shaft 2 in the longitudinal direction. For example, as shown in FIG. 1, the shaft 2 may have a reinforced section 2A and a non-reinforced section 2B in the longitudinal direction of the shaft 2. The reinforced section 2A indicates a section provided with the reinforcing material made of metal. The non-reinforced section 2B indicates a section located distal to the reinforced section 2A and not provided with the reinforcing material. With this configuration, the flexibility of the shaft 2 can be enhanced in the non-reinforced section 2B, whereby the catheter 1 can be easily curved along the shape of the body cavity. In addition, in the reinforced section 2A, the dimensional stability of the shaft 2 can be enhanced.

The shaft 2 has a side hole 4 communicating with the lumen 3. Therefore, the wire 30 can be inserted into the lumen 3 of the shaft 2 through the side hole 4. A protective tube 10 is inserted into the side hole 4. The side hole 4 is provided so as to penetrate from the outside of the shaft 2 to the lumen 3.

The side hole 4 may be provided such that the central axis of the side hole 4 coincides with the radial direction of the shaft 2. In addition, the central axis of the side hole 4 may extend from the outer side to the inner side in the radial direction from the distal side to the proximal side of the shaft 2.

The angle between the central axis of the side hole 4 and the longitudinal direction of the shaft 2 is preferably an acute angle. With this configuration, the orientation of the protective tube 10 can be more effectively controlled. Specifically, the angle between the central axis of the side hole 4 and the longitudinal direction of the shaft 2 can be 20 degrees or more, 25 degrees or more, or 30 degrees or more, or can be 60 degrees or less, 55 degrees or less, or 50 degrees or less.

The first electrode 20 functions as a measurement electrode or a reference electrode (for example, a ground electrode) at the time of potential measurement. The first electrode 20 is disposed outside the shaft 2. Examples of the shape of the first electrode 20 include a ring shape, a shape having a C-shaped cross section with a notch in the ring, and a coil shape obtained by winding a wire rod. The first electrode 20 can be placed on the shaft 2 by crimping the electrode to the shaft 2. One or more first electrodes 20 can be provided.

The first electrode 20 only needs to have conductivity, and can be made of metal or a mixture containing resin and metal. It is particularly preferable to use a conductive resin or metal such as platinum, a platinum iridium alloy, stainless steel, or tungsten as a material of the first electrode 20. It is preferable that the conductive resin contains a contrast medium such as barium sulfate or bismuth oxide in order to enable visible observation under X-ray fluoroscopy.

The wire 30 electrically connects the first electrode 20 and an external device of the catheter 1, for example, an electrocardiograph. The wire 30 is electrically connected to the first electrode 20 and extends through the protective tube 10 to the lumen 3 of the shaft 2. As a result, electrical continuity is ensured between the first electrode 20 and the electrocardiograph. Since the protective tube 10 is inserted into the side hole 4 of the shaft 2 in this manner, the shaft 2 and the wire 30 are less likely to come into contact with each other when the wire 30 is inserted, whereby the wire 30 is easily inserted into the shaft 2. In addition, even when the shaft 2 has a reinforcing material and the reinforcing material is exposed on an inner wall 5 forming the side hole 4, the wire 30 can be protected by the protective tube 10, so that damage to the wire 30 can also be prevented.

The wire 30 only needs to have conductivity, and for example, a copper wire, an iron wire, a stainless steel wire, a piano wire, a tungsten wire, a nickel titanium wire, or the like can be used. In the wire 30, a portion other than both end portions in the longitudinal direction may be covered with a covering material such as a covering tube. This makes it possible to prevent a short circuit with an adjacent member. The covering material of the wire 30 can be made of, for example, a urethane resin or an epoxy resin.

The first electrode 20 and the wire 30 can be connected by a method such as laser welding, resistance welding, or bonding with an adhesive.

The protective tube 10 is provided to facilitate insertion of the wire 30 into the lumen 3 of the shaft 2. The protective tube 10 may be inserted into a part of the side hole 4 in the depth direction, or may be inserted into the entire side hole 4 in the depth direction. The protective tube 10 is preferably a resin tube. The cross section of the protective tube 10 perpendicular to the longitudinal direction may have a circular shape, an oval shape, a polygonal shape, or a C-shape, a U-shape, or the like obtained by partly cutting any of the above shapes.

As the material constituting the protective tube 10, the description of a synthetic resin among the materials constituting the shaft 2 can be referred to. Particularly, a polyimide resin or a polyamide resin is preferable because of good dimensional stability and insertability into the side hole 4.

The protective tube 10 may have a size large enough to allow the wire 30 to be inserted into the lumen. The outer diameter of the wire 30 can be set to, for example, 0.05 mm or more, 0.1 mm or more, or 0.2 mm or more, or 1 mm or less, 0.8 mm or less, or 0.5 mm or less. Therefore, the inner diameter of the protective tube 10 may be 0.1 mm or more, 0.2 mm or more, or 0.3 mm or more, or may be 1.2 mm or less, 1 mm or less, or 0.8 mm or less.

In order to facilitate insertion of the wire 30 into the lumen of the protective tube 10, the inner diameter of the protective tube 10 is preferably larger than the outer diameter of the wire 30. Specifically, the inner diameter of the protective tube 10 is more preferably 1.1 times or more the outer diameter of the wire 30, or may be 3 times or less, 2.4 times or less, or 1.6 times or less the outer diameter of the wire 30.

The thickness of the protective tube 10 is not particularly limited as long as the protective tube 10 can keep the dimension, and can be, for example, 0.005 mm or more, 0.01 mm or more, or 0.02 mm or more. It may also be 0.1 mm or less, 0.08 mm or less, or 0.05 mm or less.

As illustrated in FIG. 2, the protective tube 10 has a first end and a second end in the longitudinal direction. The protective tube 10 has a first opening 11 at the first end of the protective tube 10 and a second opening 12 at the second end of the protective tube 10. Since both ends are opened in this manner, the wire 30 can be inserted into the protective tube 10. Preferably, the first opening 11 is located outward in the radial direction of the shaft 2 with respect to the second opening 12, and a proximal end 12A of the second opening 12 is located proximal to a proximal end 11A of the first opening 11. Since the positions of the first opening 11 and the second opening 12 are shifted in the longitudinal direction of the protective tube 10, the wire 30 is less likely to have a bent portion, so that kink of the wire 30 can be prevented.

The extending direction of the protective tube 10 is not particularly limited, but it is preferable that the protective tube 10 extends while being inclined with respect to the longitudinal direction of the shaft 2 from the viewpoint of preventing kink of the wire 30. It is more preferable that an angle between the longitudinal direction of the protective tube 10 and the longitudinal direction of the shaft 2 is an acute angle. Specifically, the angle between the longitudinal direction of the protective tube 10 and the longitudinal direction of the shaft 2 may be 20 degrees or more, 25 degrees or more, or 30 degrees or more, or may be 60 degrees or less, 55 degrees or less, or 50 degrees or less.

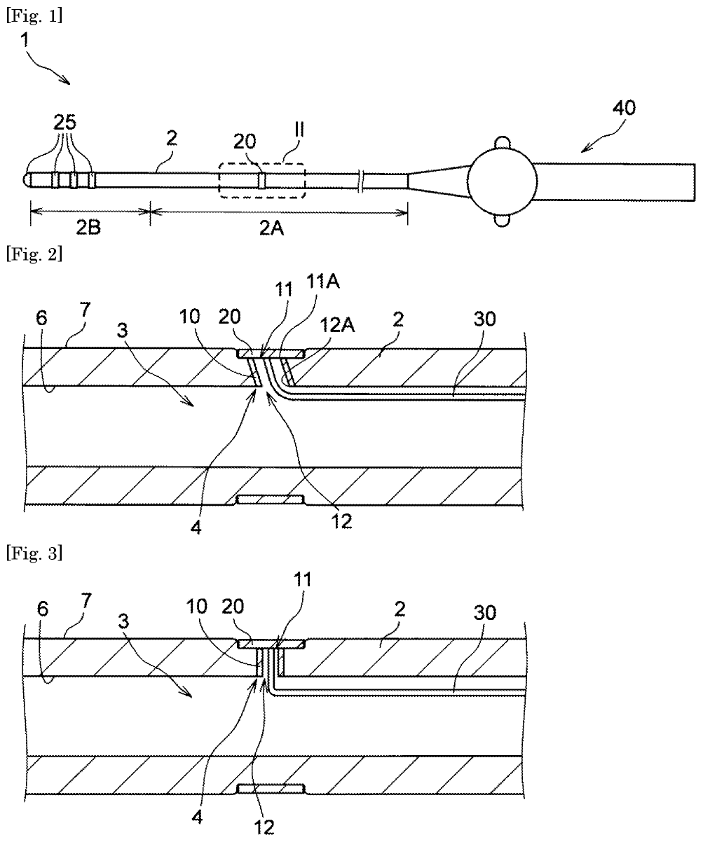

FIGS. 3 to 6 are side sectional views illustrating modifications of the catheter illustrated in FIG. 2. As illustrated in FIG. 3, the protective tube 10 may extend along the radial direction of the shaft 2. This makes it easy to insert the protective tube 10 into the side hole 4 of the shaft 2. In addition, kink of the protective tube 10 can be prevented when the protective tube 10 is inserted into the side hole 4 of the shaft 2.

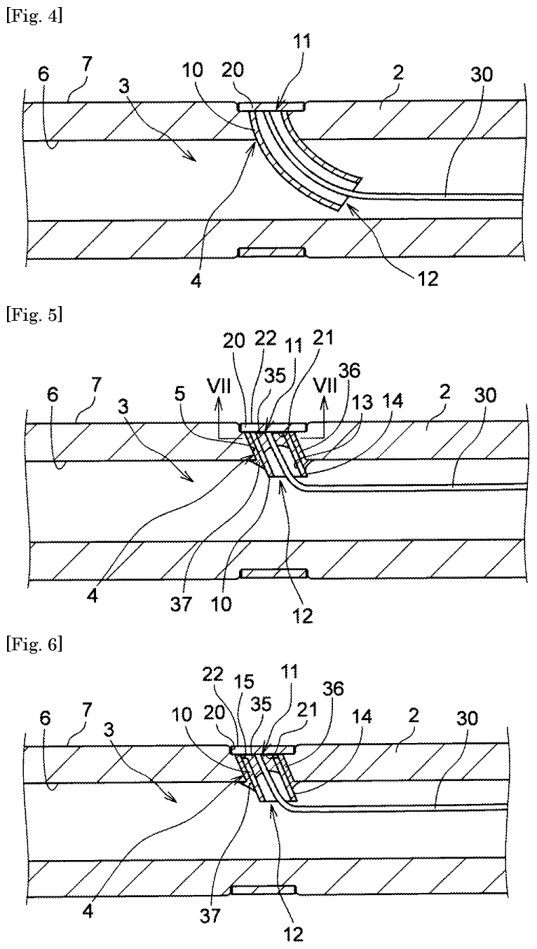

In FIG. 4, the protective tube 10 has a first end and a second end in the longitudinal direction. The protective tube 10 has a first opening 11 at the first end of the protective tube 10 and a second opening 12 at the second end of the protective tube 10. The first opening 11 is located outward in the radial direction of the shaft 2 with respect to the second opening 12. In this case, at least a part of the second opening 12 is preferably located radially inward of the inner surface 6 of the shaft 2. With this configuration, the protective tube 10 can be placed in the lumen 3 of the shaft 2, and thus, it is possible to prevent the contact between the inner surface 6 of the shaft 2 and the wire 30.

It is preferable that, as shown in FIG. 4, a portion located inward in the radial direction of the shaft 2 with respect to the inner surface 6 of the shaft 2 is longer than a portion located in the side hole 4 of the shaft 2 in the longitudinal direction of the protective tube 10. When the protective tube 10 is disposed as described above, the protective tube 10 easily extends along the longitudinal direction of the shaft 2 on the second opening 12 side of the protective tube 10. As a result, the wire 30 is less likely to have a bent portion, whereby kink of the wire 30 can be prevented.

Although not illustrated, it is preferable that the end surface of the second end of the protective tube 10 (the opening end surface of the second opening 12) is not in contact with the inner surface 6 of the shaft 2. Furthermore, it is preferable that the outer surface of the protective tube 10 on the second opening 12 side abuts on the inner surface 6 of the shaft 2. When the protective tube 10 is disposed as described above, the wire 30 is also less likely to have a bent portion, whereby kink of the wire 30 can be prevented.

The length of the portion of the protective tube 10 located inward in the radial direction of the shaft 2 with respect to the inner surface 6 of the shaft 2 is preferably longer than the inner diameter of the shaft 2. When the length of the protective tube 10 is set as described above, the protective tube 10 easily extends along the longitudinal direction of the shaft 2 on the second opening 12 side of the protective tube 10, so that the wire 30 is less likely to have a bent portion. Thus, kink of the wire 30 can be prevented.

In the following, a mode for preventing the protective tube 10 from coming out of the shaft 2 will be described. In FIG. 5, the protective tube 10 has a first end and a second end in the longitudinal direction. The protective tube 10 has a first opening 11 at the first end of the protective tube 10 and a second opening 12 at the second end of the protective tube 10. The first opening 11 is located outward in the radial direction of the shaft 2 with respect to the second opening 12. In this case, an inner adhesive 35 is preferably disposed in the protective tube 10 on the first opening 11 side. Accordingly, the first electrode 20, the wire 30, and the protective tube 10 can be firmly fixed, and thus, it is possible to prevent the protective tube 10 from coming out of the shaft 2. The first electrode 20 is preferably disposed on the side hole 4. The inner adhesive 35 may be applied up to a position inward of the inner surface 6 of the shaft 2 in the radial direction of the shaft 2.

From the viewpoint of preventing kink of the wire 30, it is preferable that the inner adhesive 35 is disposed only in a part of the protective tube 10 in the longitudinal direction without being disposed on the entire protective tube 10 in the longitudinal direction, and it is more preferable that the inner adhesive 35 is not disposed into the protective tube 10 on the second opening 12 side.

The inner adhesive 35 is preferably in contact with an inner surface 21 of the first electrode 20 and an inner surface 13 of the protective tube 10. This makes it possible to firmly fix the first electrode 20 and the protective tube 10.

As illustrated in FIG. 6, the inner adhesive 35 is preferably disposed between the inner surface 21 of the first electrode 20 and an end surface 15 of the first end of the protective tube 10. When the inner adhesive 35 is disposed as described above, the first electrode 20 and the protective tube 10 can be more firmly fixed.

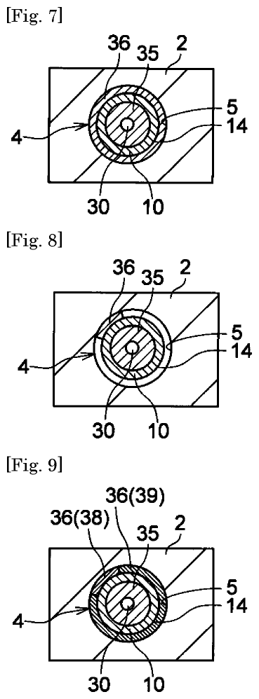

FIG. 7 is a sectional view of the catheter illustrated in FIG. 5 along a line VII-VII. As illustrated in FIG. 7, it is preferable that an outer adhesive 36 is disposed on at least a part of the protective tube 10 in the circumferential direction between the inner wall 5 forming the side hole 4 of the shaft 2 and the outer surface 14 of the protective tube 10. With this configuration, the protective tube 10 can be firmly fixed to the side hole 4 of the shaft 2, so that the protective tube 10 can be prevented from coming out of the shaft 2.

As illustrated in FIG. 7, it is more preferable that the outer adhesive 36 is disposed on the protective tube 10 throughout the circumferential direction. This makes it possible to more firmly fix the protective tube 10 to the side hole 4 of the shaft 2.

FIGS. 8 and 9 are sectional views illustrating modifications of the catheter illustrated in FIG. 7. As illustrated in FIG. 8, the outer adhesive 36 may be disposed only on a part of the protective tube 10 in the circumferential direction. When the outer adhesive 36 is disposed as described above, the protective tube 10 can also be firmly fixed to the side hole 4 of the shaft 2.

As the outer adhesive 36 and the inner adhesive 35, polyurethane-based, epoxy-based, cyano-based, or silicone-based adhesive is preferable. The outer adhesive 36 and the inner adhesive 35 may be made of the same material, or may be made of materials different from each other.

The protective tube 10 has a first end and a second end in the longitudinal direction. The protective tube 10 has a first opening 11 at the first end of the protective tube 10 and a second opening 12 at the second end of the protective tube 10. The first opening 11 is located outward in the radial direction of the shaft 2 with respect to the second opening 12. In this case, it is preferable that the inner adhesive 35 is disposed in the protective tube 10 on the first opening 11 side, and the inner adhesive 35 and the outer adhesive 36 are made of the same material. With this configuration, the inner adhesive 35 and the outer adhesive 36 are easily cured integrally, and the inner adhesive 35 and the outer adhesive 36 are satisfactorily bonded. As a result, it is possible to further prevent the protective tube 10 from coming out of the shaft 2.

As illustrated in FIG. 9, it is preferable that the outer adhesive 36 includes a first outer adhesive 38 and a second outer adhesive 39 made of materials different from each other, the first outer adhesive 38 is disposed in a first section in the circumferential direction of the protective tube 10, and the second outer adhesive 39 is disposed in a second section different from the first section in the circumferential direction of the protective tube 10. By using at least two types of adhesives as described above, it is possible to further prevent the protective tube 10 from coming out of the shaft 2.

Although not illustrated, at least two first outer adhesives 38 and two second outer adhesives 39 may be disposed in the circumferential direction of the protective tube 10. That is, at least two first sections and two second sections may be formed in the circumferential direction of the protective tube 10. In that case, the first outer adhesives 38 and the second outer adhesives 39 may be alternately arranged in the circumferential direction of the protective tube 10.

The protective tube 10 has a first end and a second end in the longitudinal direction. The protective tube 10 has a first opening 11 at the first end of the protective tube 10 and a second opening 12 at the second end of the protective tube 10. The first opening 11 is located outward in the radial direction of the shaft 2 with respect to the second opening 12. In this case, it is preferable that the inner adhesive 35 is disposed in the protective tube 10 on the first opening 11 side, and either of the first outer adhesive 38 or the second outer adhesive 39 and the inner adhesive 35 are made of the same material. With this configuration, either of the first outer adhesive 38 or the second outer adhesive 39 and the inner adhesive 35 are easily cured integrally, and they are satisfactorily bonded. As a result, it is possible to further prevent the protective tube 10 from coming out of the shaft 2.

As shown in FIGS. 5 and 6, the outer adhesive 36 is preferably disposed on the outer surface 14 of the protective tube 10 and inward of the inner surface 6 of the shaft 2 in the radial direction of the shaft 2. That is, the outer adhesive 36 preferably has a barb portion 37 on the second opening 12 side of the protective tube 10. When the outer adhesive 36 is disposed as described above, the protective tube 10 can also be firmly fixed to the shaft 2, so that the protective tube 10 can be prevented from coming out of the shaft 2.

In the radial direction of the shaft 2, the outer surface 22 of the first electrode 20 is preferably located flush with the outer surface 7 of the shaft 2 or located more inward than the outer surface 7 of the shaft 2. The exposure of the adhesive to the outer surface of the shaft 2 can be prevented. In addition, the adhesive is effectively pressure-bonded between the first electrode 20 and the shaft 2 to improve sealability, whereby the effect of preventing the inflow of liquid into the shaft 2 can be enhanced.

In FIG. 1, the shaft 2 has the reinforced section 2A and the non-reinforced section 2B in the longitudinal direction. The reinforced section 2A indicates a section provided with the reinforcing material made of metal. The non-reinforced section 2B indicates a section located distal to the reinforced section 2A and not provided with the reinforcing material. In that case, it is preferable that the first electrode 20 is located in the reinforced section 2A, and a second electrode 25 is located distal to the first electrode 20 in the non-reinforced section 2B. In that case, it is preferable that the first electrode 20 is used as a reference electrode and the second electrode 25 is used as a measurement electrode. Since the first electrode 20 is used as a reference electrode as described above, a sharp electrocardiogram waveform can be obtained. In addition, 3D mapping can be performed using the obtained measurement data, and thus, the internal structure of the site where the catheter 1 is inserted can be confirmed more accurately. The reference electrode is an electrode that provides a reference point of potential during electrode potential measurement. In order to measure potentials at various positions, it is preferable to provide a plurality of second electrodes 25 as illustrated in FIG. 1. In addition, regarding the structure, constituent material, and connection with the wire of the second electrode 25, the description of the first electrode 20 can be referred to.

Since the reinforcing material is not provided in the non-reinforced section 2B of the shaft 2 where the second electrode 25 is provided, the reinforcing material is not exposed on the inner wall 5 forming the side hole 4. Therefore, a wire (not illustrated) electrically connected to the second electrode 25 may be inserted into the lumen 3 of the shaft 2 through the side hole 4 into which the protective tube 10 is not inserted.

Next, a method for producing the catheter 1 will be described. An embodiment of the method for producing the catheter 1 according to the present invention is characterized by including: a step of opening the side hole 4 in the shaft 2 that has a first end and a second end in the longitudinal direction and that has the lumen 3 extending in the longitudinal direction, the side hole 4 communicating with the lumen 3; a step of inserting the protective tube 10 into the side hole 4; a step of applying the outer adhesive 36 to the outer surface 14 of the protective tube 10; a step of inserting the wire 30 electrically connected to the first electrode 20 into the protective tube 10; a step of inserting the inner adhesive 35 into the protective tube 10; and a step of placing the first electrode 20 above the side hole 4.

First, the shaft 2, the protective tube 10, the first electrode 20, and the wire 30 are prepared.

The shaft 2 has the first end and the second end in the longitudinal direction, and has the lumen 3 extending in the longitudinal direction. The side hole 4 communicating with the lumen 3 is opened in the shaft 2. The side hole 4 can be formed using a drilling tool such as a laser processing machine or a borer. The side hole 4 is preferably formed to penetrate from the outside of the shaft 2 to the lumen 3.

The protective tube 10 is inserted into the side hole 4. Thus, the shaft 2 and the wire 30 are less likely to come into contact with each other when the wire 30 is inserted, whereby the wire 30 is easily inserted into the shaft 2. In addition, since the wire 30 can be smoothly inserted into the shaft 2, it is also possible to prevent kink of the wire 30 and peeling of the surface covering material of the wire 30. Note that the protective tube 10 may be inserted into a part of the side hole 4 in the depth direction, or the protective tube 10 may be inserted into the entire side hole 4 or beyond the side hole 4 in the depth direction.

It is preferable to further include a step of adjusting the direction of the central axis of the side hole 4 before the step of inserting the protective tube 10 into the side hole 4. As a result, the extending direction of the protective tube 10 inserted into the side hole 4 can be adjusted. The direction of the central axis of the side hole 4 can be adjusted, for example, by inserting a bar-shaped member having an outer diameter equal to or larger than the diameter of the side hole 4 into the side hole 4. When the bar-shaped member is inserted into the side hole 4 and is moved so that the bar-shaped member is inclined with respect to the longitudinal direction of the shaft 2, the central axis of the side hole 4 can be inclined with respect to the longitudinal direction of the shaft 2. Before the step of adjusting the direction of the central axis of the side hole 4, the central axis of the side hole 4 may be parallel to the radial direction of the shaft 2. As the bar shaped member, a member having a circular or oval cross-section perpendicular to the longitudinal direction and made of metal such as stainless steel can be used, for example.

The outer adhesive 36 is applied to the outer surface 14 of the protective tube 10. As a result, the outer surface 14 of the protective tube 10 and the inner wall 5 forming the side hole 4 are joined to each other, whereby it is possible to prevent the protective tube 10 from coming out of the shaft 2. The outer adhesive 36 may be applied to a part of the protective tube 10 in the circumferential direction or may be applied to the entire protective tube 10 throughout the circumferential direction. As described above, the first outer adhesive 38 and the second outer adhesive 39 may be applied to the protective tube 10. It is preferable to perform a step of applying the outer adhesive 36 to the outer surface 14 of the protective tube 10 after the step of inserting the protective tube 10 into the side hole 4.

In the step of inserting the protective tube 10 into the side hole 4, it is preferable to insert the protective tube 10 by a first predetermined length and then pull back the protective tube 10 by a second predetermined length shorter than the first predetermined length. As a result, the barb portion 37 as illustrated in FIGS. 5 and 6 can be formed on the outer adhesive 36. Thus, the protective tube 10 is firmly fixed to the shaft 2, whereby it is possible to prevent the protective tube 10 from coming out of the shaft 2.

The wire 30 electrically connected to the first electrode 20 is inserted into the protective tube 10. The electrode and the wire 30 can be electrically connected by laser welding, resistance welding, bonding with an adhesive, or the like.

It is preferable to further include a step of removing at least a part of the portion of the protective tube 10 protruding from the shaft 2 before the step of inserting the wire 30 into the protective tube 10. As a result, when the first electrode 20 is attached to the shaft 2 by crimping or the like, a part of the protective tube 10 is less likely to be sandwiched between the first electrode 20 and the shaft 2 in the radial direction of the shaft 2. Thus, the first electrode 20 is less likely to protrude radially outward from the outer surface 7 of the shaft 2. The protective tube 10 can be removed using an edge tool such as a knife or a razor. Apart of the protective tube 10 can be cut off by the edge tool.

The inner adhesive 35 is placed in the protective tube 10. Accordingly, the first electrode 20, the protective tube 10, and the wire 30 can be firmly fixed, and thus, the protective tube 10 can be prevented from coming out of the shaft 2.

The first electrode 20 is disposed above the side hole 4. After the first electrode 20 is disposed, the first electrode 20 can be fixed to the shaft 2 by crimping the first electrode 20 to the shaft 2. It is preferable to perform the step of placing the inner adhesive 35 into the protective tube 10 before the step of placing the first electrode 20 above the side hole 4.

This application claims the benefit of the priority date of Japanese patent application No. 2019-117003 filed on Jun. 25, 2019. All of the contents of the Japanese patent application No. 2019-117003 filed on Jun. 25, 2019 are incorporated by reference herein.

### REFERENCE SIGNS LIST

1: Catheter
2: Shaft
2A: Reinforced section
2B: Non-reinforced section
3: Lumen
4: Side hole
5: Inner wall forming the side hole
6: Inner surface
7: Outer surface
10: Protective tube
11: First opening
11A: Proximal end of the first opening
12: Second opening
12A: Proximal end of the second opening
13: Inner surface of the protective tube
14: Outer surface of the protective tube
15: End surface of the first end of the protective tube
20: First electrode
21: Inner surface
22: Outer surface
25: Second electrode
30: Wire
35: Inner adhesive
36: Outer adhesive
37: Barb portion
38: First outer adhesive
39: Second outer adhesive

1. A catheter comprising:
a shaft having a first end and a second end in a longitudinal direction and a lumen extending in the longitudinal direction, the shaft having a side hole that penetrates from an inner surface to an outer surface of the shaft;
a protective tube inserted in the side hole;
a first electrode disposed outside of the shaft; and
a wire electrically connected to the first electrode and extending in the lumen of the shaft through the protective tube.

2. The catheter according to claim 1, wherein
the protective tube has a first end and a second end in a longitudinal direction of the protective tube,
the protective tube has a first opening at the first end of the protective tube and a second opening at the second end of the protective tube,
the protective tube is disposed in the side hole such that the second opening is located at a lumen side of the shaft and the first opening is located at an outer surface side of the shaft, and a proximal end of the second opening is located at a positon closer to a proximal end of the catheter than a proximal end of the first opening.

3. The catheter according to claim 1, wherein the protective tube inwardly extends along a radial direction of the shaft.

4. The catheter according to claim 1, wherein
the protective tube has a first end and a second end in a longitudinal direction of the protective tube,
the protective tube has a first opening at the first end of the protective tube and a second opening at the second end of the protective tube,
the protective tube is disposed in the side hole such that the first opening is located at an outer surface side of the shaft, and at least a part of the second end of the protective tube inwardly protrudes from an inner surface of the shaft in the radial direction, whereby at least a part of the second opening is located at an inner position than the inner surface of the shaft.

5. The catheter according to claim 1, wherein
the protective tube has a first end and a second end in a longitudinal direction of the protective tube,
the protective tube has a first opening at the first end of the protective tube and a second opening at the second end of the protective tube,
the protective tube is disposed in the side hole such that the first opening is located at an outer surface side of the shaft, and
an inner adhesive is disposed in the protective tube on a first opening side to fix the wire.

6. The catheter according to claim 5, wherein the inner adhesive is disposed between an inner surface of the first electrode and an end surface of the first end of the protective tube.

7. The catheter according to claim 1, further comprising an outer adhesive disposed between an inner wall forming the side hole of the shaft and an outer surface of the protective tube, the outer adhesive being disposed on at least a part of a circumferential direction of the protective tube.

8. The catheter according to claim 7, wherein the outer adhesive is disposed around an entire circumference of the protective tube.

9. The catheter according to claim 7, wherein the outer adhesive is disposed on the outer surface of the protective tube and located radially inward of the shaft than the inner surface of the shaft.

10. The catheter according to claim 7, wherein
the protective tube has a first end and a second end in a longitudinal direction of the protective tube,
the protective tube has a first opening at the first end of the protective tube and a second opening at the second end of the protective tube,
the protective tube is disposed in the side hole such that the first opening is located at an outer surface side of the shaft,
an inner adhesive is disposed in the protective tube on the first opening side, and
the inner adhesive and the outer adhesive are made of a same material.

11. The catheter according to claim 7, wherein
the outer adhesive has a first outer adhesive and a second outer adhesive made of different materials,
the first outer adhesive is disposed on a first section in the circumferential direction of the protective tube, and
the second outer adhesive is disposed on a second section other than the first section in the circumferential direction of the protective tube.

12. The catheter according to claim 11, wherein
the protective tube has a first end and a second end in a longitudinal direction of the protective tube,
the protective tube has a first opening at the first end of the protective tube and a second opening at the second end of the protective tube,
the first opening is located radially outward of the shaft than the second opening,
an inner adhesive is disposed in the protective tube on the first opening side, and
the inner adhesive and one of the first outer adhesive and the second outer adhesive are made of a same material.

13. The catheter according to claim 1, wherein an outer surface of the first electrode is disposed on a same plane as the outer surface of the shaft or disposed inward of the outer surface of the shaft in the radial direction of the shaft.

14. The catheter according to claim 1, wherein an angle between a central axis of the side hole and the longitudinal direction of the shaft is an acute angle.

15. The catheter according to claim 1, wherein a length of a portion of the protective tube inwardly extruding from the inner surface of the shaft is longer than an inner diameter of the shaft.

16. The catheter according to claim 1, wherein
the shaft has a reinforced section and a non-reinforced section in the longitudinal direction of the shaft,
a reinforcing material made of metal is disposed in the reinforced section,
the non-reinforced section is located at a distal side of the catheter, the reinforced section is located at a proximal side of the catheter, and the reinforcing material is not disposed in the non-reinforced section,
the first electrode is disposed in the reinforced section, and
a second electrode is disposed in the non-reinforced section.

17. A method for producing a catheter comprising:
making a side hole in a shaft having a lumen so that the side hole penetrates from an outer surface to an inner surface of the shaft, the shaft having a first end and a second end in a longitudinal direction, the lumen extending in the longitudinal direction;
inserting a protective tube into the side hole;
applying an outer adhesive to an outer surface of the protective tube;
inserting a wire electrically connected to a first electrode into the protective tube;
inserting an inner adhesive into the protective tube; and
disposing the first electrode on the side hole.

18. The method for producing a catheter according to claim 17, further comprising adjusting a direction of a central axis of the side hole before inserting the protective tube into the side hole.

19. The method for producing a catheter according to claim 17, further comprising removing at least a part of a portion of the protective tube protruding from the shaft before inserting the wire into the protective tube.

20. The method for producing a catheter according to claim 17, wherein in the step of inserting the protective tube into the side hole, the protection tube is pulled back by a second predetermined length shorter than a first predetermined length after inserting the protective tube by the first predetermined length.
